(19) United States
(12) Patent Application Publication
(10) Pub. No.: US 2008/0046082 A1
(43) Pub. Date: Feb. 21, 2008

(54) INTERVERTEBRAL DISC AND NUCLEUS PROSTHESIS

(76) Inventor: Casey K. Lee, Florham Park, NJ (US)

Correspondence Address: MILES & STOCKBRIDGE PC, 1751 PINNACLE DRIVE, SUITE 500, MCLEAN, VA, 22102-3833, US

(21) Appl. No.: 11/830,690

(22) Filed: Jul. 30, 2007

Related U.S. Application Data
(62) Division of application No. 10/779,873, filed on Feb. 18, 2004, now abandoned.

(51) Int. Cl. A61F 2/44

(52) U.S. Cl. 623/17.16; 623/17.12; 623/17.15

(57) ABSTRACT

A prosthetic implant for replacing a nucleus pulposus of an intervertebral disk includes upper and lower endwalls of discoid cross-section, each having an antero-posterior diameter less than its transverse diameter, and an hourglass-shaped sidewall connecting the peripheries of the upper endwall and lower endwall to enclose an interior volume filled with a substantially incompressible liquid or soft plastic material. A total prosthesis for replacing the entire human intervertebral disk has an annular core made of a first biocompatible polymer surrounding a central cavity, transitional plates affixed respectively to the upper and lower surfaces of the annular core, the upper and lower transitional plates being made of a second biocompatible material having an elastic modulus greater than that of the first biocompatible polymer, and upper and lower endplates adapted to contact adjacent vertebrae and affixed respectively to the upper and lower transitional plates.

### RELATIONSHIP TO OTHER APPLICATIONS

This application is a division of U.S. application Ser. No. 10/779,873, filed Feb. 18, 2004. This application also claims the benefit of the priority of U.S. Provisional Patent Application No. 60/487,605, filed Jul. 17, 2003, the entire disclosure of which is incorporated herein by reference. This application also claims the benefit of the priority of U.S. Provisional Patent Application No. 60/524,902 filed Nov. 26, 2003.

### BACKGROUND OF THE INVENTION

1. Field of the Invention

This invention relates to prostheses for replacing structures of the human spine and more particularly to prostheses for replacing an intervertebral disk and/or the nucleus pulposus thereof.

2. Brief Description of the Prior Art

Lower back pain is a very common disorder and is responsible for extensive morbidity and lost time at work. The prevalence rate of low back pain is very high, affecting approximately 80% of general population at some time. Although most patients experience the painful symptoms only occasionally and recover fully, approximately 10% of these patients experience chronic and disabling low back pain in spite of various medical treatments.

The most common cause of chronic disabling low back pain is degenerative disk disease (DDD). Spinal fusion has been an effective treatment method for chronic disabling low back pain that is not responding to non-surgical treatments. It is estimated that approximately 350,000 spinal fusion procedures are being performed in the USA per year. The most common indication for spinal fusion (51% of all spinal fusion cases) has been chronic low back pain caused by various stages of DDD (internal disk derangements, disk herniation, discogenic instability and spinal stenosis). Only recently, new technologies of disk replacement and nucleus replacement have emerged for treatment of discogenic pain.

Although spinal fusion procedure has been the standard for surgical treatment of chronic low back pain caused by DDD, it has presented significant problems:

a) Obtaining successful fusion has not been free of problems. The successful fusion rate has remained almost constant at an average of 85% success in spite of development of various new techniques and instruments. Furthermore, the clinical success rate after spinal fusion has remained at an average of 75% during the past 20-30 years.

b) The average time for recuperation after spinal fusion is about 15 months.

c) Spinal fusion eliminates the motion and shock absorption function of the fused spinal motion segment. This, in turn, is the main cause of accelerated degeneration of the spinal motion segment adjacent to the fusion. To achieve the same or better results as spinal fusion, various types of artificial disk prostheses have been developed, as discussed more fully below, and some are in clinical trials in humans.

Anatomy and Biomechanics of the Intervertebral Disk:

The intervertebral disk is a complex joint having three distinct parts: vertebral endplates, the nucleus pulposus and the annulus fibrosus. The disk is a weight-bearing joint that transmits the load from one vertebral body to the next. The disk is the major stabilizing structure of the spinal column, at the same time allowing motion in three perpendicular planes. Motion in the sagittal plane (flexion/extension) is the greatest (8°-15°). Motion in the coronal plane (lateral bending) and in horizontal plane (torsion) is less. The disk also has a shock absorption function by reason of its viscoelastic properties.

The load-bearing function of the disk is accomplished by transferring the compressive load from the vertebral endplates to the annulus fibrosus by “hoop stress” through the incompressible fluid-filled nucleus pulposus. An intact nucleus pulposus, by reason of its incompressible nature, is the key for this load transfer mechanism and maintenance of the disk height. The nucleus pulposus functions as the center of rotation for motion. The center of rotation is not a fixed one but rather an instant center of rotation. On flexion, it moves posteriorly, and it moves anteriorly on extension. The nucleus pulposus normally occupies 20% to 40% of the cross section of the disk, and it becomes larger in older age and in degenerative conditions. It is made of loosely arranged type II collagen and proteoglycans. The nucleus pulposus contains approximately 80% water by weight in young and healthy disks, but the water content decreases with older age and with degeneration. Retention of such a high content of water is essential for the nucleus pulposus to function as a weight transfer medium through the annulus by “hoop stress”. The nucleus cavity of the normal disk is not a spherical or oval shape. The anatomical cross-section, MRI and discogram clearly demonstrate that the nucleus cavity is made of two chambers (upper and lower) and these two chambers are connected by an “hour-glass” shaped neck at the middle in both anterior-posterior and medial-lateral projections (See FIG. 4).

The annulus fibrosus is the most important structure for the weight bearing function and stability of the disk. The annulus fibrosus is made of 8-12 layers of laminated collagen fibers, mostly type I, running at an angle of +/−30° to the endplates. The annulus fibrosus has a varying thickness in different sections of the disk. It is thicker anteriorly and thinner posteriorly. The cross-section of the annulus fibrosus has a greater area at the mid level of the disk than at the upper and lower ends of the annulus closer to the vertebral endplates, thus forming a cavity having a cross-sectional profile of a “dumb-bell” or “hourglass” shape (See FIG. 4). The wall of the annulus is thicker at the mid-level than near the vertebral endplates especially in the anterior region of the disk. Consequently, the nucleus pulposus is not spherical or ovoid as illustrated in many anatomy books and implemented in almost of all prior designs for a disk prosthesis or nucleus pulposus prosthesis. This relationship of the “dumb-bell” or “hourglass” shape of the nucleus pulposus and the complementary shape of the annulus fibrosus probably has a significant role in the stress transmission and motion patterns of the disk. The annulus fibrosus bulges inward as well as outward on compression bending in the normal disk. In the degenerated disk, the complementary relationship between the “hourglass” structure of the nucleus pulposus and the complementary cavity in the annulus fibrosis disappears.

The relatively large cross-sectional area of the nucleus pulposus at its contact surface with the vertebral endplates is essential for wider stress distribution that prevents vertebral endplate failure. The contact surface area between the disk and the vertebral end plate, the applied load, and bone mineral density are key factors related to failure of the vertebral endplates (subsidence). For a given patient, the applied load (body weight) and the bone mineral density are fixed, but the contact surface area may be variable depending on the prosthetic design.

On flexion, the anterior column of the annulus will buckle outward and inward under the compression-flexion load, and the posterior column of the annulus will be elongated without a significant posterior bulge because of the characteristic anatomic configurations of the annulus and the nucleus pulposus as described above. The presence of a spherical or an oval-shaped prosthesis in the nucleus cavity will produce a very different behavior. On compression, stress will be equally distributed around a spherical or an oval shaped cavity filled with isotropic fluids or material. This will cause stress concentration at a small contact surface area between the endplates and the prosthesis. On compression-flexion, the anterior column of the annulus will produce a force that pushes the prosthesis posteriorly causing excessive posterior wall bulge or extrusion of the prosthesis. The “hourglass” shape of the nucleus pulposus and the complementary shape of the annulus also help to stabilize the nucleus within the disk throughout the ranges of motion of the spinal motion segment.

Vertebral Endplates:

The vertebral endplate is made of a very thin layer of condensed cancellous bone (bony endplate) and a cartilaginous layer (cartilaginous endplate). The endplate is a weight bearing transition structure between the vertebral body and the disk. It is an important passageway for fluids and nutrients between the vertebral bone and the disk. The morphology, i.e., shape and contour, of the vertebral endplates and its clinical significance have escaped the interest of scientists, such as anatomists and biomechanicians as well as of clinicians and surgeons. Consequently, the biomechanical and clinical significance of the endplate and associated structures is poorly understood.

Abnormal changes of the vertebral endplates and surrounding bone are frequently observed in degenerative disk disease. Actual failure of the vertebral endplates (compression/burst fracture) is observed in trauma. Subsidence of a bone graft, intervertebral fusion device, or disk prosthesis through the endplates into vertebral bone has been a frequently reported problem in the reconstructive surgery of the lumbo-sacral spine. Such problems as subsidence, sclerosis, bone marrow edema, and contour changes are due to abnormal stress patterns between vertebral bone and the disk.

Artificial Disc Prostheses:

Artificial disc prostheses may be divided into two general types, the total disc prosthesis and the nucleus prosthesis. The total disc prosthesis is designed for replacing the entire disc, while the nucleus prosthesis is designed for replacing only the nucleus pulposus.

The nucleus prosthesis is designed to replace only the nucleus part of the disk in order to restore the biomechanics of the degenerated disc. There are several different types of designs of the nucleus prosthesis. Some of them were clinically tested in humans, and significant problems were found, such as, e.g., extrusion, migration, subsidence and/or adverse changes at the vertebral endplates. Some types of nucleus prosthesis require removal of a significant amount of the annulus fibrosus for surgical implant. This causes further destabilization of the disc, because the nucleus prosthesis is not designed specifically to restore the function of the annulus fibrosus. Most of the nucleus prostheses are indicated for the earlier phase of disk regeneration where there is no or minimum disruption of the annulus fibrosus. The current designs of the nucleus prosthesis use three different approaches to reproduce the biomechanical effect of incompressible hydrostatic pressure within the nucleus cavity: One approach employs structures with one or more cavities (such as balloons or bladders) which are filled and inflated with fluids, gas or other injectable materials after they are placed into the disc by a minimally invasive surgical technique. Another approach is implanting dehydrated or partially hydrated hydrophilic materials in a balloon or fibrous jacket into the nucleus cavity by an open surgical exposure where the implanted material becomes hydrated. Yet another approach is to inject a polymerizable biomaterial into the nucleus cavity where it will be polymerized into an appropriate shape.

However these prior designs present certain problems. In spherical or oval designs the area of contact between the prosthesis and the vertebral endplate tends to be relatively small, thereby producing stress concentration, subsidence, and/or endplate reaction. Spherical balloon prostheses may cause a posterior bulge of the disk wall upon flexion, thereby producing abnormal stress on the posterior annulus, which can make it prone to extrusion or migration. Consequently, these designs are indicated only for very minimum degeneration of the disc with intact annulus or with very minimal annular disruption.

Another design for an intervertebral disk prosthesis is a “capsule” prosthesis. Such a prosthesis is indicated for a wider range of disc degeneration including some annular disruption. However, the surgical approaches for implant of this type of device produce further disruption of the annulus, and the stability of the device within the disc tends to be poor. Furthermore, such a prosthesis does not restore the biomechanics of the natural intervertebral disk. It does not have enough contact surface area, which causes subsidence and post-operative changes in the endplates, and it tends to produce non-physiologic patterns of motion because the center of rotation and the instant axis of rotation are quite different from the normal.

Other problems arise when fluids, gases or biomaterials are placed within an inflatable nucleus prosthesis. Such materials function as isotropic in nature. A pressure applied to one point is exerted equally at other parts of the material. Typically, when the device is inflated, only a small surface area will come in contact with the vertebral endplates causing stress concentration. Furthermore, the wall of such a device will have a tendency to bulge more toward the minimally resistant area of the annulus fibrosus such as a posterior annular fissure.

Accordingly, a need has continued to exist for an intervertebral disk prosthesis that is not subject to the deficiencies of the hitherto available prostheses.

### SUMMARY OF THE INVENTION

A prosthetic implant for replacing a nucleus pulposus of an intervertebral disk includes:

upper and lower endwalls of discoid cross-section each having an antero-posterior diameter less than its transverse diameter; and

an hourglass-shaped sidewall connecting the peripheries of the upper endwall and lower endwall to enclose an interior volume filled with a substantially incompressible liquid or soft plastic material.

A total prosthesis for replacing the entire human intervertebral disk intervertebral disk comprises,

an annular core surrounding a central cavity having upper and lower and side surfaces and made of a first biocompatible material shaped and sized to approximate the annulus fibrosus of a natural intervertebral disk, the first biocompatible material being an elastomer having a elastic modulus approximating that of the annulus fibrosus of the natural human intervertebral disk;

upper and lower transitional plates affixed respectively to the upper and lower surfaces of the annular core, the upper and lower transitional plates being made of a second biocompatible material having a durometer hardness greater than that of the first biocompatible polymer; and

upper and lower endplates adapted to contact adjacent vertebrae and affixed respectively to the upper and lower transitional plates.

Accordingly, it is an object of the invention to provide a prosthesis for replacing a human intervertebral disk.

A further object is to provide a prosthesis for replacing a human intervertebral disk wherein the prosthesis accurately corresponds to the structure and function of a human intervertebral disk.

A further object is to provide a prosthesis for a human intervertebral disk which includes a structure to replace the nucleus pulposus.

A further object is to provide a prosthesis for a human intervertebral disk which includes an hourglass-shaped structure to replace the nucleus pulposus.

A further object is to provide a prosthesis for replacing the nucleus pulposus of a human intervertebral disk.

A further object is to provide a prosthesis for replacing the nucleus pulposus of a human intervertebral disk having a shape and function that mimics the natural nucleus pulposus.

A further object is to provide a prosthesis for replacing the nucleus pulposus of a human intervertebral disk having an hourglass shape, resembling that of the natural human nucleus pulposus.

A further object is to provide a prosthesis for replacing the nucleus pulposus of a human intervertebral disk that can be implanted using minimally invasive surgical techniques.

A further object is to provide a prosthesis for replacing the nucleus pulposus of a human intervertebral disk that can be collapsed for insertion by minimally invasive surgical techniques and inflated after implantation.

Other objects of the invention will become apparent from the description of the invention which follows.

### BRIEF DESCRIPTION OF THE DRAWINGS

FIG. 1A is a schematic side view of a pair of normal human vertebrae with the intervertebral disk shown in cross-section, wherein the vertebrae are in their normal position.

FIG. 1B is a somewhat enlarged cross-section of the intervertebral disk of FIG. 1A.

FIG. 1C is a view similar to that of FIG. 1A with the structures shown in a spinal column in flexion.

FIG. 1D is a view similar to that of FIG. 1A with the structures shown in a spinal column in extension.

FIG. 2A is a plan view of the nucleus pulposus prosthesis of the invention.

FIG. 2B is a front elevational view of the nucleus pulposus prosthesis of the invention.

FIG. 2C is a front elevational cross-section view of the nucleus pulposus prosthesis of the invention.

FIG. 2D is a left side lateral elevational view of the nucleus pulposus prosthesis of the invention.

FIG. 3A is a perspective view of the nucleus pulposus prosthesis of the invention shown in phantom as implanted within a natural annulus fibrosus.

FIG. 3B is an anterior elevational cross-sectional view of the nucleus pulposus prosthesis in place within an annulus fibrosus of an intervertebral disk.

FIG. 3C is a left-side lateral elevational view, in partial cross-section, of the nucleus pulposus prosthesis in place within an annulus fibrosus of an intervertebral disk.

FIG. 4 is a discogram showing an x-ray view of a normal human intervertebral disk located between two vertebrae with the nucleus pulposus being visualized with injected contrast medium.

FIG. 5 is a graph showing the scanned profile of vertebral endplates of adjacent vertebrae.

FIG. 6 is a top plan view of the metal endplate used in the total intervertebral disk prosthesis of the invention.

FIG. 7 is a top plan view of the anterior extension plate used with the metal endplate of FIG. 6.

FIG. 8 is a front elevational view of the metal endplate of FIG. 6.

FIG. 9 is an exploded cross-sectional view of the total disk prosthesis of the invention taken along the line 9-9 in FIG. 6 and FIG. 7.

FIG. 10 is a cross-sectional view of the total disk prosthesis of FIG. 9 as assembled.

FIG. 11 is a lateral cross-sectional view of one embodiment of the total prosthesis of the invention as implanted between two vertebrae.

FIG. 12 is a top plan view of the core portion of the total disk prosthesis of FIG. 6.

FIG. 13 is a front elevational view plan view of the core portion of the total disk prosthesis of FIG. 6 as indicated by the line 13-13 in FIG. 12.

FIG. 14 is a front elevational cross-sectional view of the core portion of FIG. 12 taken along the line 14-14 in FIG. 12.

FIG. 15 is a top plan view of the polymer annulus of the core portion of FIG. 13 as indicated by the line 15-15 in FIG. 13.

FIG. 16 is a lateral cross-sectional view of a variation of the total disk prosthesis of FIGS. 6-15.

FIG. 17 is a lateral elevational view of the total disk prosthesis of FIGS. 6-15 as assembled.

FIG. 18 is a lateral elevational view of a variation of the total disk prosthesis of FIG. 17 using a tightened cable to fasten certain components together.

FIG. 19 is a detail view of the cable fastening structure of the total disk prosthesis of FIG. 18

FIG. 20 is a top plan view of a transition plate used in an alternate embodiment of the invention.

FIG. 21 is a left side elevational view of the transition plate of FIG. 20.

FIG. 22 is a front elevational view of the transition plate of FIG. 20.

FIG. 23 is a bottom plan view of the transition plate of FIG. 20.

FIG. 24 is a top plan view of an endplate used with the transition plate of FIG. 20.

FIG. 25 is a left side elevational view of the endplate of FIG. 24.

FIG. 26 is a left side elevational cross sectional view of the endplate of FIG. 24 taken along the line 25-25 in FIG. 24.

FIG. 27 is a front elevational view of the endplate of FIG. 24.

FIG. 28 is a bottom plan view of the endplate of FIG. 24.

FIG. 29 is a front elevational view of an assembly of the transition plate of FIG. 20 and the endplate of FIG. 24.

FIG. 30 is a left side elevational view of the assembly of FIG. 29.

### DETAILED DESCRIPTION OF THE INVENTION AND PREFERRED EMBODIMENTS

The invention includes a prosthesis for replacing the nucleus pulposus of a human intervertebral disk and a prosthesis for replacing an entire intervertebral disk.

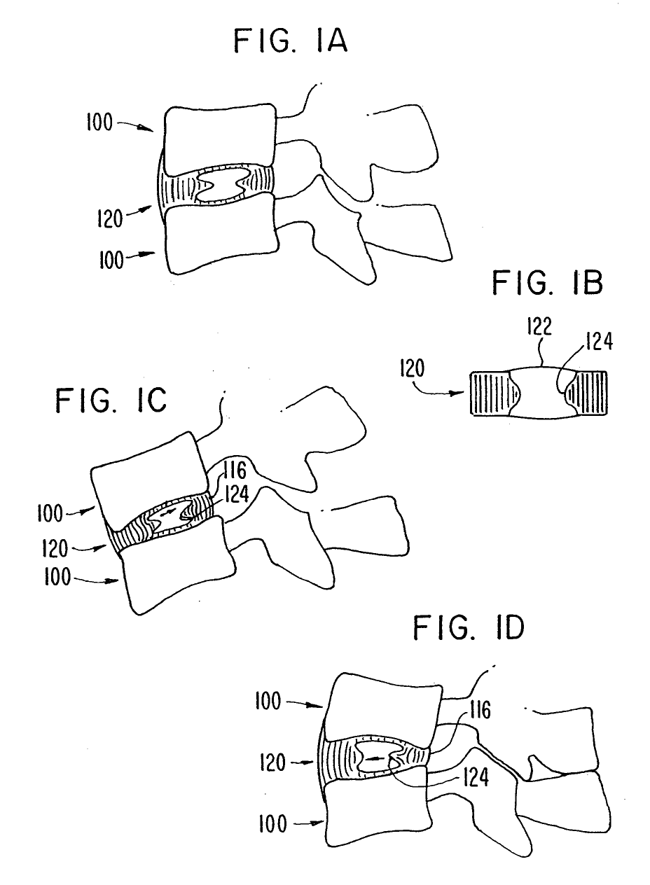

FIGS. 1A-1D schematically illustrate the natural human intervertebral disk 120, in cross-section, positioned between two vertebrae 100. FIG. 1A shows the configuration of the intervertebral disk 120 when the vertebral column of the spine is in a neutral position. FIG. 1B is a somewhat enlarged cross-section of the intervertebral disk 120, showing the natural nucleus pulposus 122 surrounded by the natural annulus fibrosus 116. The hourglass shape of the natural nucleus pulposus 122 produced by the inwardly bulging inner wall 124 of the natural annulus fibrosus can be seen. FIG. 1C shows the configuration of the intervertebral disk when the spine is in flexion compressing the anterior edge of the annulus fibrosus 116, causing the internal wall 124 to bulge inward, and the posterior edge of the annulus fibrosus 116 is stretched. The result, as shown, is a posterior movement of the center of rotation. Conversely, as shown in FIG. 1D, when the spine is in extension, the posterior edge of the annulus fibrosus 116 is compressed, and the anterior edge is stretched, causing the center of rotation to move anteriorly.

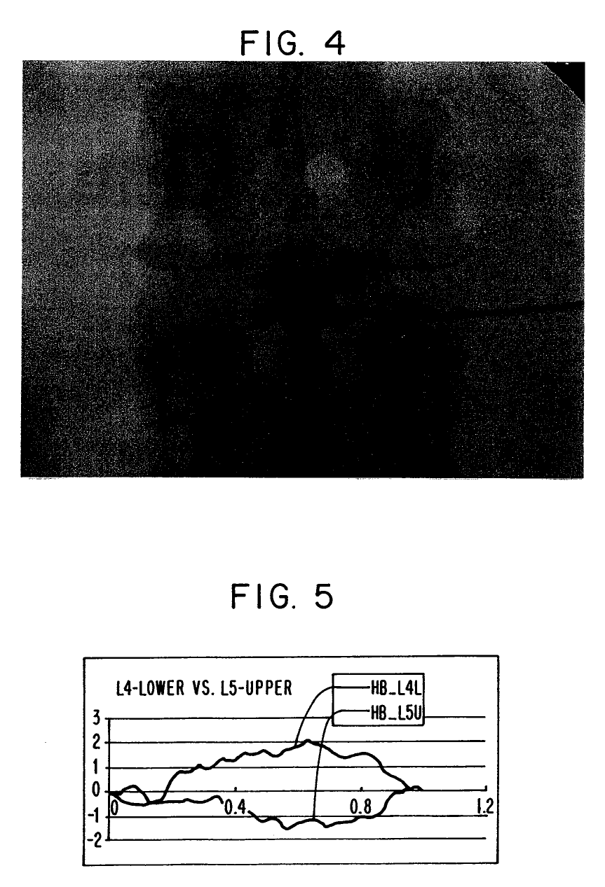

The shape of the internal wall of the annulus fibrosus 116 and the hourglass shape of the nucleus pulposus within the annulus fibrosus is illustrated in the discogram of a natural intervertebral disk shown in FIG. 4, wherein the structures are visualized by x-rays using an appropriate contrast medium.

The Nucleus Pulposus Prosthesis:

The nucleus pulposus prosthesis according to the invention is an endoprosthesis for replacement of a diseased or degenerated natural nucleus pulposus, after removal thereof, and for partial replacement of a minimally to moderately disrupted annulus fibrosus of an intervertebral disk. The device is designed to articulate with the natural cartilagenous vertebral endplates. The device comprises a thin, flexible wall having a shape designed to mimic the shape of the natural nucleus pulposus and enclosing a hollow cavity that can be filled with a liquid, gas or soft synthetic polymer to mimic the viscoelastic behavior of the natural nucleus pulposus. It can be considered to be an inflatable balloon having a specific shape and contour when it is fully inflated. It comprises three elements: two endplate sections and a “dumb-bell” or “hourglass” shaped middle section. The device may be implanted as fully inflated or may be implanted as the collapsed form and inflated after implantation. Two lateral stabilizing cords may be provided. One of these cords may provide an access route to the nucleus prosthesis cavity for inflation.

When the nucleus pulposus prosthesis is fully inflated, the endplate sections (upper and lower) are generally similar in shape, and each is configured to have a dome-shape convex toward the vertebral bone with a specific curvature to conform to the host vertebral endplate with which it is in contact. The average maximum depth for the lower endplate is about 2.0 mm, and the average maximum depth of the upper endplate is about 1.2 mm (typically ranging from about 0.6-1.5 mm). The endplate sections of this prosthesis are typically made of a thicker layer or a harder durometer of biomaterial than the mid-section lateral wall. The endplates may also have fiber reinforcement. The endplate sections are preferably made stiffer than the lateral walls of the mid section to maintain the specific degree of the dome shape contour when the prosthesis is inflated. In a cross-section or plan view the endplate sections of the nucleus pulposus prosthesis present a “discoid” shape. The contact surface area of the endplate disk, i.e., the area in contact with the vertebral endplate, is typically approximately 30%-60% of the vertebral endplate cross-sectional surface area. The size of the contact surface area of the endplate sections of the device in an individual patient will be determined by the size of the host vertebral bone and by the degree of nucleus/disk degeneration. A larger size will typically be chosen for a more seriously degenerated disc. In contrast to conventional spherical or oval shaped prostheses for replacement of the nucleus pulposus, the nucleus pulposus prosthesis of this invention can provide a wide range of endplate contact surface area to accommodate variable degrees of disc degeneration. As the degree of disc degeneration progresses, the nucleus cavity becomes larger, and the weight bearing ability of the annulus fibrosus decreases. When the prosthesis is placed in the nucleus cavity, the maximum depth of the convexity of the endplates of the prosthesis is at 60% posteriorly on the antero-posterior (A-P) dimension of the vertebral endplates. The top or apex of the dome will be mid position on medial-lateral (M-L) dimension.

The mid-section is given an hourglass shape to accommodate the normal anatomy of the annulus and to avoid excessive bulging of the sidewall on bending. The thickness of the walls of the hourglass may vary in anterior, posterior or lateral portions of the walls to produce desired shapes and contours. This configuration of the mid-section allows the annulus fibrosus to bulge inwardly in the same patterns as in the normal disc during the range of motion. This contour of the mid-section also stabilizes the nucleus prosthesis during flexion-extension and lateral bending under the compressive load by interlocking of the hourglass contour of the prosthesis with the complementary shape (also described as a “vase” shape) of the annulus (wider thickness at the mid-section). The device has a valve mechanism attached for inflation of the cavity. An extension tube from the valve may be brought out to the exterior part of the disc through the annulus wall for easy access. Two extension tubes may be used, one on each side, and they may function as stabilizing structures for the prosthesis within the disk when the outside end is secured to the exterior wall of the disk.

The shape of the endplate section and the “hourglass” shaped mid section will preferably be made with different thickness and/or hardness grades of an elastomeric polymer such as a polycarbonate thermoplastic-polyurethane blend.

The device is preferably collapsible and so that it can be rolled into a tube for insertion through a blunt hole in the postero-lateral annulus. After implantation into the nucleus cavity it is inflated with fluid or biocompatible polymer to produce the intended shape and contour. The intended shape and contour is achieved by molding the device from a biocompatible polymer with various thickness, hardness or stiffness in different sections of the device. The deformation characteristics of the device under compression-bending and axial loading will be controlled by the differential stiffness of various sections of the device.

The nucleus pulposus prosthesis can be implanted using a percutaneous approach through serial cannulas or through a minimally invasive surgical approach. Bi-portal scopes may be introduced into the nucleus cavity, one from each side, after dilation of the annular hole with a series of probes and cannulas of increasing diameter inserted through the posterior-lateral aspect of the disc. The upper and lower nucleus cavities are cleaned by removal of degenerated/disrupted materials, leaving the mid-section of the annulus intact. The nucleus pulposus prosthesis device is introduced through the cannula and is subsequently inflated with biocompatible fluid or appropriate biocompatible viscoelastic polymer material. The nucleus pulposus prosthesis may be stabilized further by one or more non-absorbable retention sutures, cords or tubes that are attached to the device and brought outside of the disc for anchoring to structures, e.g., bone or appropriate soft tissue, outside of the disc. Preferably two such sutures, cords or tubes are used, one on each side of the nucleus pulposus prosthesis. One or more of such stabilizing elements can be a tube through which the nucleus pulposus prosthesis is inflated.

Preferred embodiments of the nucleus pulposus prosthesis are designed to facilitate as natural function as possible of the entire intervertebral disk, whether formed by the remaining natural annulus fibrosus together with the nucleus pulposus prosthesis or by a prosthetic annulus fibrosus in a total intervertebral disk replacement.

Accordingly, the nucleus pulposus prosthesis of the invention is preferably designed to have a form and contours, when it is fully inflated, that match the form and contours of the natural nucleus pulposus. This is accomplished by making different portions of the prosthesis with different viscoelastic properties. For example, different regions of the prosthesis can be molded with different thickness or hardness of materials for different sections of the device, e.g., different portions of the wall, as will be discussed more fully below.

The top and the bottom plates of the nucleus pulposus prosthesis preferably have a contour that conforms as closely as possible to that of the vertebral endplates with which they are in contact. Such a design provides the largest possible contact surface area between the nucleus pulposus prosthesis and the vertebral endplates, which minimizes stress concentration at the interface and provides maximum protection against subsidence of the prosthesis.

The endplates are discoid in shape in transverse cross-section, and are preferably molded to have a shape and contour that matches the vertebral surface with which they come into contact. In particular, the endplates of the nucleus pulposus prosthesis are preferably provided in various sizes to match the mating vertebral endplate. Typical sizes of the prosthesis endplates will have cross-sectional areas ranging from about 30% to about 60% of the cross-sectional area of the mating vertebral endplate. However, the prothesis endplates may be larger if necessary to achieve satisfactory biomechanical properties in the surgically repaired intervertebral plate. Prosthesis endplates of larger size are indicated for more advanced disc degeneration where the nucleus cavity is larger and annular disruption is greater. In such cases, because the disrupted and/or degenerated annulus fibrosus has a reduced weight bearing capability, a relatively larger contact surface area between the vertebral endplate and the prosthesis endplate is needed to prevent vertebral endplate failure.

Preferably the endplates of the nucleus pulposus prosthesis are made stiffer than the walls connecting them, e.g., by making them thicker, by making them from a harder plastic material, i.e., a material having a greater durometer value, or by fiber reinforcement. More preferably, the prosthesis endplates are made sufficiently rigid to ensure even distribution of stress at the interface between the prosthesis and the vertebral endplates during compression or compression-bending loads.

Preferably each nucleus pulposus prosthesis endplate has a contour matched to the corresponding contour of the mating vertebral endplate. Typically, the depth of the convexity of the nucleus pulposus prosthesis endplate toward the vertebral endplate will average about 1.2 mm (ranging from about 0.7 mm to about 1.5 mm) for the upper end plates and averaging about 2.0 mm (ranging from about 1.5 mm to about 2.5 mm) for the lower endplates. The maximum depth of the convexity is located generally at the mid-position of the right-left diameter and about 60% posterior from the anterior rim along the anterior-posterior diameter. The skilled practitioner will understand that the particular dimensions of a particular prosthesis are preferably adapted for the best match to the vertebral plates of the patient receiving the prosthesis.

The middle section of the nucleus pulposus prosthesis has the characteristic “dumb-bell” or “hourglass” configuration designed to facilitate restoring the biomechanics of the intervertebral disk as closely as possible to normal. In this respect it is believed that the prosthesis of this invention more closely approximates the normal function than previously known designs. This hourglass configuration also provides stability of the prosthesis within the disk preventing it from migration and/or extrusion. Preferably, the concavity of the lateral wall of the mid section to form the “hourglass” differs at anterior, posterior, and lateral walls. The lateral walls have less concavity than the anterior wall. Accordingly, the anterior and posterior walls tend to deform more than the lateral walls during bending because the vertebrae have a greater range of motion in flexion/extension than in lateral bending of the particular spinal segment. Furthermore, because the anterior wall of the annulus fibrosus is much thicker than the posterior wall, it needs more room for displacement during compression-flexion.

The nucleus pulposus prosthesis of the invention is preferably collapsible in order to allow it to be implanted by minimally invasive surgical approaches. After implantation into the disk cavity, such a collapsible prosthesis is inflated by injecting a filling material, e.g., a liquid or fluid material, polymerizable or curable materials in a fluid state, synthetic hyaluronic acid, or the like. The filler may be introduced by any conventional technique, e.g., using a syringe and needle or other cannula, or through one or more extension tubes attached to the lateral wall of the prosthesis that are sealed off by a valve mechanism or in-situ sealing with biomaterial after the filling of the prosthesis has been completed. If such extension tubes are used in a preferred embodiment, a pair of such tubes, or equivalent cords, or the like, preferably one on each side, may be secured to anatomical structures outside of the disk in order to further stabilize the prosthesis.

The nucleus pulposus prosthesis of the invention has a wider indication for discs with variable degrees of degeneration than hitherto known prostheses. Unlike any spherical or oval shaped prosthesis, wherein the contact between the prosthesis and endplate typically occurs over a somewhat restricted area, the nucleus pulposus prosthesis of the invention permits a wide range of the endplate contact surface area to accommodate variable degrees of disc degeneration.

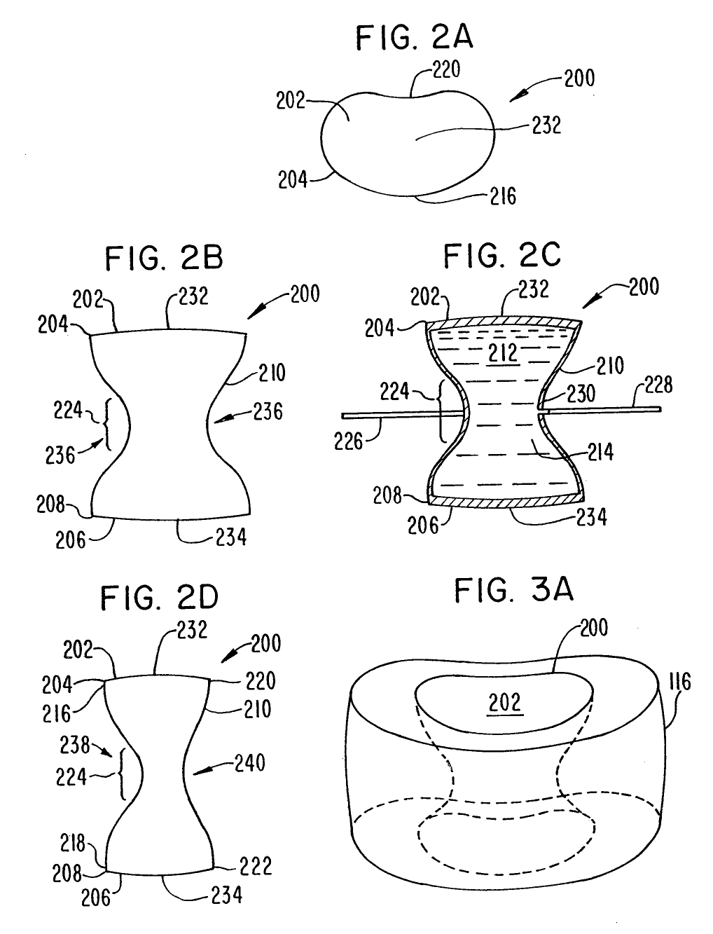

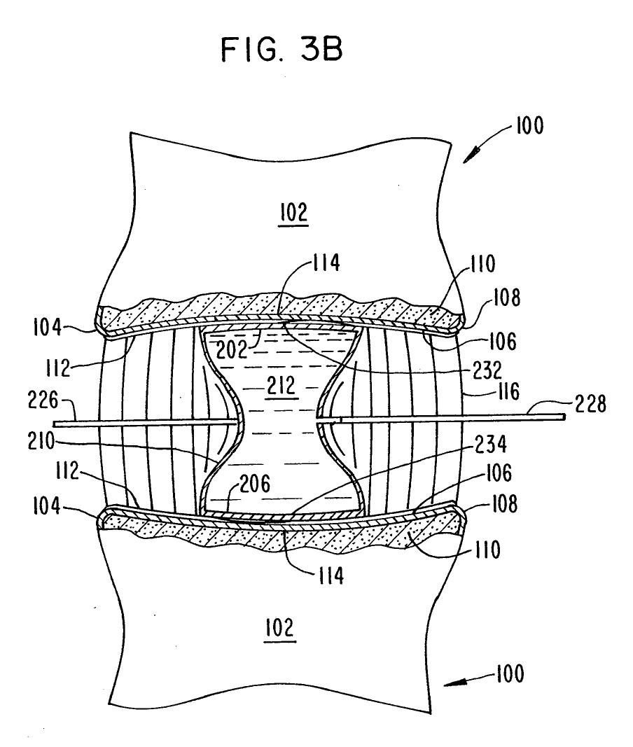

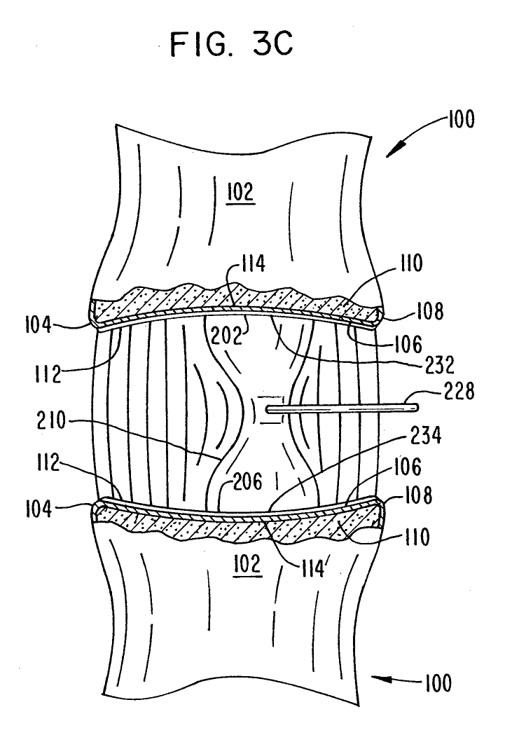

An embodiment of the nucleus pulposus prosthesis of the invention is illustrated in FIGS. 2A-2D and FIGS. 3A-3C.

FIG. 2A illustrates a top plan view of the nucleus pulposus prosthesis 200. FIG. 2B illustrates a front elevational view of the nucleus pulposus prosthesis 200 of the invention, and FIG. 2C illustrates a front elevational cross-sectional view of the nucleus pulposus prosthesis 200. FIG. 2D illustrates a left side elevational view of the nucleus pulposus prosthesis 200. The nucleus pulposus prosthesis 200 comprises a top wall or endplate 202, having a top wall periphery 204, a bottom wall or endplate 206, having a bottom wall periphery 208, and a sidewall 210 extending between the top endwall periphery 204 and the bottom endwall periphery 208, to enclose an internal volume 212 filled with a suitable generally incompressible fluid or viscoelastic material 214, as described above. The top endwall 202 and bottom endwall 206 have a plan shape that generally duplicates the horizontal cross section of the natural nucleus pulposus at its interface with the vertebral plates of the superior and inferior vertebrae, respectively. Accordingly, the plan shape of the top endwall 202 and bottom endwall 206 is a somewhat flattened disk, having a greater lateral (i.e., side-to-side) dimension than an antero-posterior dimension (the dimension from the anterior edge 216, 218 to the posterior edge 220, 222 of the endwall). The posterior edge of the plan shape typically is recurved to mimic, at least approximately, the natural cross section of the nucleus pulposus. The top endwall 202 and bottom endwall 206 are typically and preferably of the same shape and size. However, it is not excluded that they may differ somewhat in shape and size in order to accommodate the needs of a particular patient.

The sidewall 210 of the nucleus pulposus prosthesis 200 has an hourglass or dumbbell shape, to mimic, at least approximately, the natural shape of the nucleus pulposus, and thereby provide a substitute for the natural nucleus pulposus. The shape of the natural nucleus pulposus is illustrated, for example in the discogram shown in FIG. 4. Accordingly, the superior and inferior portions of lateral wall 210, adjacent to and attached to the upper endwall 202 and lower endwall 206, have cross sectional dimensions approximating the corresponding dimensions of the top wall 202 and bottom wall 208, respectively, while a middle or waist portion 224 has cross-sectional dimensions substantially less than those of the superior and inferior portions of the lateral wall 210. The hourglass shape of the nucleus pulposus prosthesis cooperates with the natural shape of the annulus fibrosus to provide an accurate replacement of the support and flexibility provided by the natural nucleus pulposus of the intervertebral disk.

Although the nucleus pulposus prosthesis 200 of the invention may be manufactured and filled with a generally incompressible material and implanted by conventional open surgical techniques, it is preferred that the nucleus pulposus 200 be installed empty by being rolled or otherwise collapsed and introduced through a tube into the cavity formed by removing the natural nucleus pulposus. After introduction, the nucleus pulposus prosthesis 200 is unfolded and inflated by filling with a fluid material introduced through a cannula. The material may be a liquid or polymerizable material that will polymerize in situ to form a suitable filler for the nucleus pulposus prosthesis.

In order to support the nucleus pulposus prosthesis 200 in its designed position within the intervertebral disk, it may be provided with one or more cords or sutures 226, 228 that can be secured to anatomical structures outside the intervertebral disk to stabilize it. In order to provide secure attachment points for the cords 226, 228 a thickened portion 230 of the sidewall 210 may be provided in the waist region 224

Typically the concave curvature of the lateral aspects 236 of the sidewall 210 is less than that of the anterior portion 238 and posterior portion 240 of the sidewall 210.

The nucleus pulposus prosthesis 200 is filled with an incompressible, yet fluid or flexible material 214. Such liquid materials as aqueous normal saline solution, a biocompatible oil, a synthetic hyaluronic acid/proteoglycan composition, and a soft biocompatible synthetic polymer are representative of suitable filling materials. The soft solid materials should preferably have a modulus in the range of 0-4 Mpa. In particular the soft biocompatible synthetic polymer preferably has a modulus in the range of 0-1 Mpa.

FIGS. 3A-3C illustrate the nucleus pulposus prosthesis 200 of the invention in position within the intervertebral disk. FIG. 3A shows a phantom perspective view of the nucleus pulposus 200 showing its position within the annulus fibrosus 116 of an intervertebral disk. FIG. 3B shows an anterior view in partial cross-section of the nucleus pulposus 200 positioned within an intervertebral disk 112 between superior and inferior vertebrae 100. Each vertebra comprises a vertebral body 102, having a vertebral rim (or epiphyseal ring) 104 and a vertebral endplate 106. The ends of the vertebrae nearest the intervertebral disk are partially cut away to show its structure comprising a thin layer 108 of dense bone backed by the cancellous bone 110 of the interior of the body of the vertebra 100. Each of the vertebral endplates 106 is covered with a thin layer of cartilage 112. The concave curvature of the vertebral endplates provides each of them with an apex 114, i.e., the point of greatest distance from a line defined by the edges of the vertebral rims 104. The apex 114 of each of the vertebral endplates 106 is located generally midway between the sides of the vertebrae 100, as shown in FIG. 2B, and generally about 60% of the distance between the anterior edge 116 and the posterior edge 118 of the vertebral rim 104, as shown in FIG. 3C. Each of the endwalls 202, 206 of the nucleus pulposus prosthesis 200 has a corresponding apex 232, 234, which is defined by the greatest distance from a line defined by the periphery of the endwalls 202, 206. The apexes 232, 234, are is located to contact the corresponding apexes 114 of the vertebral endplates 106.

The Total Disk Prosthesis:

The total disk prosthesis of the invention has been developed to provide an elastomeric core having biomechanical characteristics, i.e., motion, shock absorption, stability, and the like, similar to those of the annulus and nucleus of the natural intervertebral disk. The prosthesis incorporates prosthetic vertebral endplates with a specific shape and contour based on a morphometric study of the natural vertebral endplate, and incorporating structures for fixation at the interface between the vertebral bone and the prosthetic endplates, as well as structures and configuration for articulation at the interface between the elastomeric disc prosthesis core and the prosthetic endplates.

In order to provide accurate information regarding shape and contour of the natural vertebral endplates, a new morphometric study of the lumbosacral vertebral endplates was conducted.

Hitherto information regarding the exact shape, contour and the geometry of the lumbosacral vertebral bone has not been readily available. Accordingly, a morphometric study of the vertebral endplates of the adult human lumbar spine was conducted by using a highly reliable measuring technique. The contour of the vertebral endplates was determined by scanning with a non-contact laser sensor (LMI DynaVision SPR-04 laser sensor, manufactured by LMI Technologies, Inc., Delta, British Columbia). The data from a typical scan of opposed vertebral endplates facing an intervertebral space filled by an intervertebral disk is shown in FIG. 5.

The results of this study provided new information on morphometric characteristics of the human lumbar vertebral endplates. In particular, the method of this study has gone beyond previous studies in providing a very accurate continuous tracing of the endplate's contour both in anterior-posterior and right-left dimension. In general, the vertebral endplate has a concave curvature toward the vertebral body, and the concavity of the curvature of the lower endplate is different from that of the upper endplate. The results of the measurements for vertebrae in the lumbosacral region, specifically for the lower endplate of the third lumbar vertebra (L3L), the upper and lower endplates of the fourth and fifth lumbar vertebrae (L4U, L4L, L5U, L5L), and the upper plate of the first sacral vertebra (S1U), are presented in Table 1 below.

Lumbosacral Vertebral Endplates Curvature

| | n | age | range | L3L | L4U | L4L | L5U | L5L | S1U |
|---|---|---|---|---|---|---|---|---|---|
| Male | 7 | 36 | (25-40) | 1.54 | 1.16 | 1.9 | 1.4 | 1.87 | 1.13 |
| Female | 9 | 25 | (25-40) | 1.9 | 1.04 | 1.8 | 0.6 | 1.87 | 0.29 |
| Mean | 16 | 35.5 | (25-40) | 1.72 | 1.1 | 1.85 | 1 | 1.87 | 0.71 |

Vertex: 60% anterior-posterior (A-P); 50% mediolateral (M-L)

The maximum depth of the curvature of the lower vertebral endplates of L3, L4 and L5 was 1.8 mm in average, and that of the upper endplates of L4 and L5 was 0.93 mm in average. The vertex of the curvature was located at the middle on the coronal plane and at 60% in the average from the anterior margin to the posterior margin.

The total disc prosthesis of the invention comprises three sections: a polymer disk core and two endplates.

The polymer disk core is comprised of three elements: a polymer annulus and two transitional endplates. The polymer annulus has an outer wall preferably made of a biocompatible polymer. The outer wall is shaped and sized to provide an operative substitute for the natural annulus fibrosus. Accordingly, the general transverse cross-section of the polymer core is disk-shaped having a lateral dimension somewhat greater than its anterior-posterior (A-P) dimension and somewhat flattened on its posterior aspect. The outer wall has a radial thickness generally approximating the radial thickness of the natural annulus fibrosus. The outer wall surrounds a central cavity intended to be filled with a material that will provide a substitute for the natural nucleus pulposus, as discussed in more detail below.

Preferably, the outer wall is configured to provide a central cavity with an “hourglass”, or “dumbell” shaped cross-sectional area, i.e., having a radial thickness that is greater at the midpoint between upper and lower end surfaces than adjacent to the upper and lower surfaces. The inner “hourglass” shaped cavity that substitutes for the natural nucleus pulposus is filled with fluid, oil, soft biomaterial or synthetic hyaluronic acid, and the wall of the cavity is shaped to confine the filling material in an “hourglass” shape. Accordingly, the outer wall of the prosthetic annulus has an appropriate thickness and stiffness to match the biomechanical characteristics as provided by the natural annulus fibrosus in the intact intervertebral disk. The central cavity of the prosthetic annulus, which provides the “hourglass” shape of the natural nucleus pulposus, has a size in the range of about 20%-50% of the volume of the polymer core, and has an e-value of 0-4 Mpa. The annulus part occupies 50%-80% of the polymer core, and has an e-value of 3-16 Mpa. The material filling the “hourglass” shaped nucleus cavity may be the same type of material as in the annulus, but with softer consistency, or it may be a different type of material. The annulus part of the polymer core is affixed to the upper and lower transitional polymer endplates, and the nucleus cavity is thereby sealed off completely by the annulus and endplates. The transitional polymer endplates may be molded to the polymer annulus, or may be adhesively secured to the polymer annulus by a suitable biocompatible adhesive. The nucleus cavity may be filled at the time that the transitional polymer endplates are molded, sealed, or the like, to the polymer annulus, or it may be filled after the transitional endplates are sealed to the polymer annulus through a port that will be sealed off after the filling process.

The nucleus cavity may alternatively be cylindrical, oval or discoid shape, and may be filled with a fluid, such as an aqueous or oily material, a soft synthetic or natural biomaterial, e.g., synthetic hyaluronic acid, or a soft synthetic polymeric material of a type different from that used for the polymer annulus.

In the construction of the intervertebral disk prosthesis of the invention it is necessary to provide a suitable interface between the hard metal endplate and the elastomeric polymer core component of the disk prosthesis. Such an interface must deal with problems presented by 1) possible stress concentration at or near the interface due to a huge difference in stiffness between the metal plate and the synthetic polymer core, and 2) attachment/fixation of the polymer core to the metal endplate.

According to the invention a transition polymer plate is used between the hard metal endplate and softer synthetic polymer core.

The polymer transition plate is made of a polymer having a hardness with a value between that of the hard metal endplate and the softer polymer core. The polymer transition plate is molded or otherwise securely affixed to the polymer annular component of the core in order to provide a smooth transition of stress, without any stress concentration. Preferably, the material used for the transition polymer plate is relatively hard (Shore A 100-D 65), so that it allows a secure mechanical fixation to the metal endplate, or allows free gliding motion at the contact surface with the metal endplate as in a total hip and knee prosthesis.

The top and bottom polymer endplates of the core are made of a material that is harder than the material of the annulus portion of the core, and have a dome shape for contact with domed metal endplates. The transitional endplates are preferably made of a material of the same chemical class as the annulus part of the polymer core, such as an aromatic and/or aliphatic polycarbonate thermoplastic-polyurethane blend, but are relatively hard, (100A-65D durometer). The thickness of the posterior end of the polymer transitional plates is 1-3 mm and the thickness of the anterior wall is 4-7 mm. The inside surface of the transitional polymer plate facing the synthetic polymer annulus is preferably flat. The difference between the thickness at the anterior and posterior edges of the transition polymer endplate orients the metal endplates at a suitable lordotic angle (5-15 degrees). The metal endplate is convex toward the vertebral bony endplates with the following preferred specific dimensions based on the results of the above-described morphometric study of the natural vertebral endplates. Both the polymer and metal endplates are discoid in transverse shape and preferably have closely matching opposing surfaces at the interface therebetween.

The maximum depth of the curvature of the dome of the lower endplate is an average of 2 mm (1.5-2.5 mm), and that of the upper endplate is an average of 1.2 mm (0.7-1.5 mm). The maximum depth is preferably located at a point 60% posteriorly between the anterior and posterior margins of the vertebral endplates, and generally midway between the right and left margins. Accordingly, the polymer core has a generally discoid cross-section and has a surface area generally matching that of the matching contact surface of the metal endplates. The central nucleus cavity of the polymer core may be inflated prior to surgical implant or after surgical implant, as indicated above.

The metal endplates are preferably configured to have the best match in shape and contour to the vertebral endplates with which they come into contact, based on the results of the new morphometric study. Preferred specific features of the metal endplates are as follows: 1) The upper endplate, which faces the lower vertebral endplate of the superior vertebra, has a matching convexity with a maximum depth of the curvature in the range of 1.5 mm-2.5 mm, located at the midline in the coronal plane (right-left) and at 60% posteriorly from the anterior edge in the sagittal plane (anterior-posterior). 2) The lower endplate, which faces the upper vertebral endplate of the inferior vertebra, has a matching convexity with the maximum depth of the curvature in the range of 0.6-2.0 mm, located at the midline in the coronal plane and at 60% posteriorly from the anterior edge in the sagittal plane. In order to have the best congruous fit, the natural vertebral endplate is reamed to match the metal endplate to provide a smoother contact surface.

The shape of the metal endplate is similar to the natural vertebral endplate, i.e., the average size of the curved portion of the metal endplate is about 2.5 cm (2.0-3.0 cm) for the minor diameter (anterior-posterior) and about 3.0 cm (2.5-3.5 cm) for the major diameter (right-left). The endplate is sized to provide a contact surface area in an individual patient, i.e., the area of the vertebral endplate that is contacted by the endplate of the prosthesis, of about 30% to 100% of the cross-sectional area of the vertebral endplate. Preferably the contact area is about 30-80% of the vertebral endplate cross-sectional area. The metal endplate preferably has a generally vertical fin oriented antero-posteriorly and positioned on the midline of the plate at its anterior edge. This fin is intended to fit into a recess formed in the anterior aspect of the vertebral bone to improve the fixation of the metal endplate to the vertebra. The fin may be provided with a slot to receive a mating locating projection on an extension plate, that serves to locate the extension plate, as discussed below.

The metal endplates are made of any suitably strong and biocompatible metal, e.g., a Co—Cr alloy or a titanium alloy. The outer surface of the top and bottom endplates facing the vertebral bone is provided with a porous texture to promote secure fixation by reason of bone ingrowth.

The metal endplate and the transitional polymer endplate may have free gliding motion with respect to each other. In order to provide a smooth and specially hardened surface of the transition polymer core endplate to facilitate such smooth gliding motion, the metal-contacting surface of the polymer transition plate may be treated with a conventional ionization treatment.

Alternatively, the endplates and polymer core component may be fixed securely to one another by one of several methods, as discussed below.

Each endplate system (metal endplate and transitional polymer endplate in contact therewith) may use a two-component structure (metal endplate and transition polymer plate) or a three-component structure (metal endplate, one transition polymer endplate and a metal anterior extension plate).

In each structure (two-component or three-component), the posterior margin of the metal endplate may have a generally perpendicular wall curving away from the vertebral bone to engage the posterior edge of the polymer transitional plate (e.g., as tongue and groove).

Alternatively, in a recessed-posterior embodiment, the metal endplate and the transitional polymer endplate may have a “step cut” fit at the posterior one-fourth to one-half of the prosthesis. In such an embodiment, the posterior portion of the transitional plate has a recessed portion in its outer surface, extending from a notch or step, located at a position ¼ to ½ of the antero-posterior diameter forward of the posterior margin of the transitional plate, to the posterior margin. Thus the recessed portion extends over the posterior¼ to ½ of the antero-posterior diameter of the transitional plate, and the outer surface of the recessed portion is generally and preferably parallel to the inner surface of the transitional plate. The step typically extends from the left margin to the right margin of the transitional plate. It may be a straight step extending generally parallel to the side-to-side (lateral or coronal) diameter of the transitional plate, or it may be curved, i.e., it may be concave or convex with respect to the anterior portion of the transitional plate. Furthermore, the face of the step may extend generally perpendicular to the outer surface of the recessed portion (and the inner surface of the transitional plate), or it may be inclined in an antero-posterior direction. That is, the step, viewed from a lateral aspect, may present a beveled profile or an undercut profile.

In the recessed posterior embodiment, the prosthesis endplate, typically made of metal, has a thicker posterior portion, with a step in its inner surface corresponding to, and generally matching, the step in the outer surface of the transitional plate. Preferably the step in the outer surface of the transitional plate transitional plate and the step in the inner surface of the prosthesis endplate are undercut so as to provide a positive mechanical connection between the transition plate and the prosthesis endplate. The positive mechanical interlock provided by the matching transverse steps in the transition plate and prosthesis endplate provides a strong control to minimize or eliminate torsional rotation between the plates. Furthermore, in this embodiment, there is no need for a curved hook extension at the posterior margin of the prosthesis (metal) endplate, and the posterior margin of the transitional plate need not extend beyond the posterior margin of the annulus. Accordingly, this arrangement provides a prosthesis that is well adapted for positioning in the intervertebral space with the vertex of the metal endplates located at the preferred location, i.e., on the anteroposterior diameter of the vertebra, about 60% of the diameter posterior to the anterior edge of the vertebra. It is especially useful for implantation in certain patients having an intervertebral disk with a small antero-posterior diameter

In the two-component structure the metal endplate has a curved anterior perpendicular wall covering ½ or ⅓ of the anterior wall of the transitional polymer plate. In the two-component structure, the anterior portion of the metal plate extends anteriorly beyond the curved portion of the metal endplate and is continuous therewith (one piece). This anterior area faces the dense peripheral rim of the vertebral body. The generally flat area of the anterior extension has an average anterior-posterior dimension of about 0.8 cm; however, the antero-posterior diameter may vary from zero (i.e., no anterior extension) to about 1.2 cm. The average width of the anterior extension portion is about 3.0 cm at the posterior portion with gradual tapering anteriorly to match the contour of the anterior margin of the vertebral endplate. The metal and transitional polymer plates may be fixed together by one or more screws fastening the anterior perpendicular wall of the metal endplate to the anterior wall of the polymer transition plate, e.g. one screw on each side. Alternatively the metal endplate and transitional polymer endplate may be fastened by clips tensioned by one or more wires or cables, as discussed below. Additional fixation may be effected by screws engaging lateral appendages of the metal and transition polymer endplates.

In another embodiment, the metal endplate and transition polymer endplate may be firmly engaged together by a snap-fit effected by spring clips at the lateral and/or anterior margins of the metal endplate. These spring clips may act by themselves or may be supplemented by screws or by cable tensioning of the spring clips.

The three-component structure comprises the convex shaped metal endplate (the main metal endplate) and an anterior extension plate that is separate from the main metal endplate. The total contact surface area between the metal endplate and the vertebral bone is in the range between 50% and 80% of the surface of the vertebral endplate. The anterior extension plate, which extends generally horizontally, has a curved wall perpendicular to the anterior extension plate, projecting away from the vertebral bone. This perpendicular wall has a curvature matching that of the anterior wall of the polymer transition plate of the core. The anterior extension plate is also provided with a vertical fin projecting toward the vertebral bone at the midline, running in an anterior-posterior direction and extending posterior to the posterior margin of the extension plate to engage a mating socket in a corresponding fin on the main metal endplate. The fin extends for a total anterior-posterior distance of about ⅓ to ½ of the anterior-posterior of the vertebral body. The horizontal anterior extension plate has a screw holes on each side of the midline for fixation of the plate to the vertebral endplate by screws extending from the disk space into the bone. The perpendicular curved wall of the anterior extension plate may also have screw holes, e.g., one on each side of the midline, to fix the anterior plate to the transitional polymer endplate. The transitional polymer endplate may have female screw threads molded therein.

The three-component structure of the total disc prosthesis is adapted for removal and replacement of the core portion thereof if revision surgery should be required. If revision or replacement of one of the currently available disc prostheses is necessary, removing all components of the previously implanted prosthesis presents a serious problem. Almost all current designs of total disc prostheses have metal endplates fixed to the vertebral bone with the inter-positioning member(s) locked or secured to the metal plates. Removal of such a prosthesis generally requires destruction of the prosthesis and disengagement of the metal endplates from the bone, because there is no provision for repair within the implantation site. Evidently, such surgery is difficult and may cause additional trauma.

The anterior extension plate may have alternative or additional fixation to the polymer transition plate and the metal endplate by engagement of fins and/or a screw/wire/cable locking mechanism attached on each side of the prosthesis.

In one embodiment of the prosthesis of the invention, lateral extension blocks are provided on the metal endplate, polymer transition plate and the anterior extension plate, the lateral extension blocks having holes for screws/cable/wire on each side of the disc prosthesis that are lined up when the endplates and core three disk are assembled during the surgical procedure. Screws, wire, cable or a self-locking device will secure all three components tightly together.

In this embodiment design the metal endplate may have curved wings at the periphery for snap fitting of the polymer transitional plate, and additional securing of these components may be made with wire or cable around the peripheral wings as indicated above.

In this embodiment of the total disk prosthesis the polymer core can be removed without disturbing the metal endplates. To remove the core, the anterior extension plate is separated from the rest of dome shaped metal endplate, but may remain fixed to the transition polymer endplate with screws and/or wires and/or cable as indicated above. Alternatively, the anterior extension plates may be detached from the dome shaped main metal endplate to provide an access window for removal or replacement of the polymer core component without explanting the main metal endplates. After insertion of a new polymer core, the anterior extension plates may be reattached with wire/cable or screws as indicated above. Consequently, this embodiment of the total disk prosthesis of the invention allows easy revision of the disc prosthesis.

It should be noted that the excellent fit between the metal endplates and the vertebral endplates provided by the total disk prosthesis of the invention bone, with shape and contour of the prosthetic endplates matched to the natural vertebral endplates for the most congruous fit, is conducive to uniform stress transmission and long-term in-vivo stability of the device.

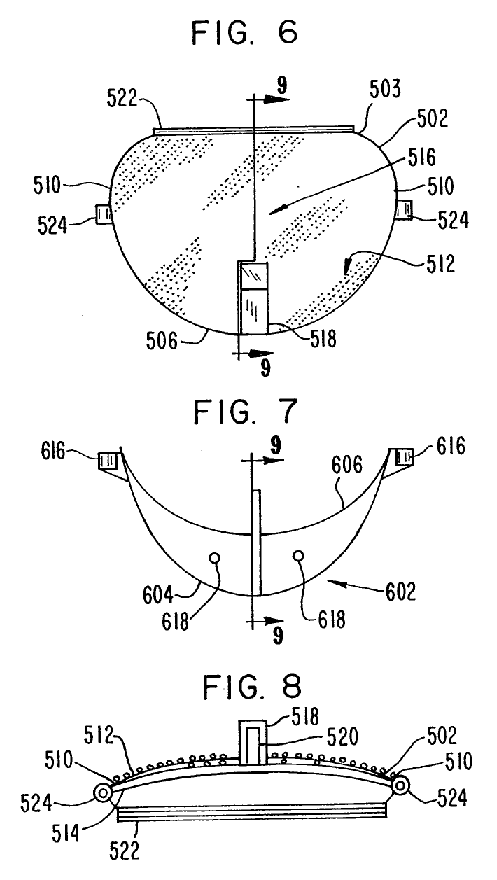

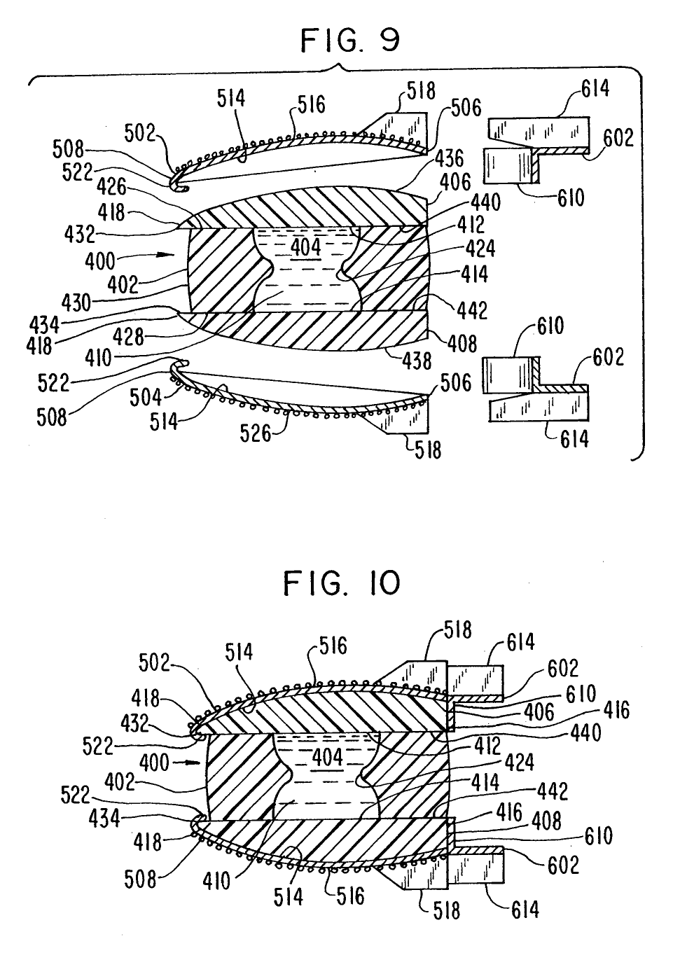

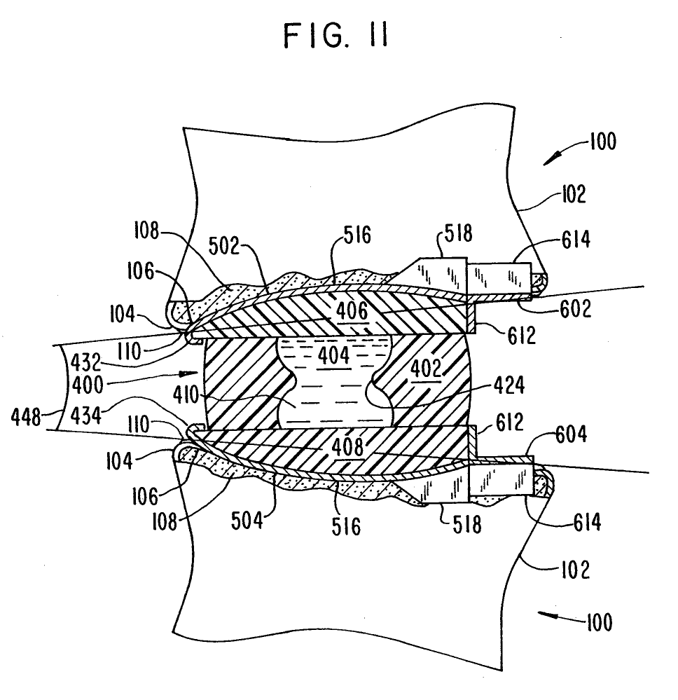

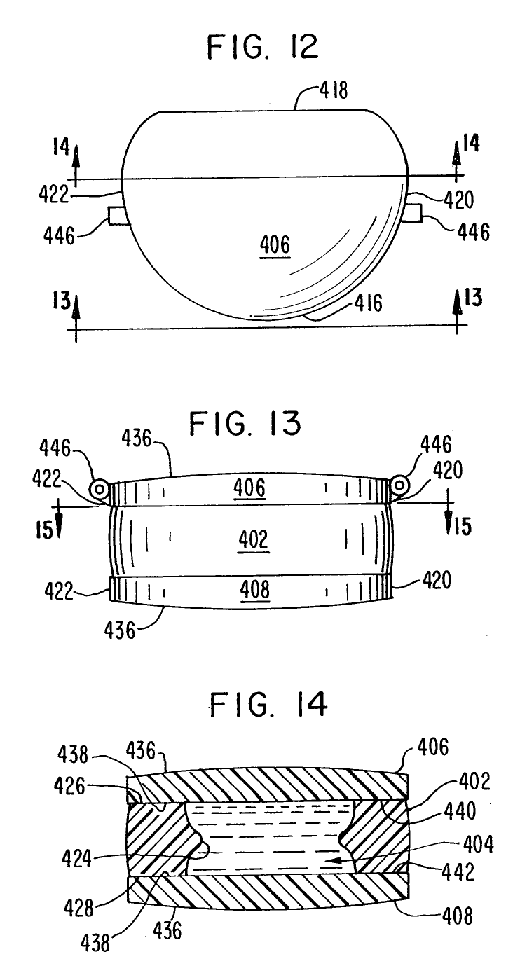

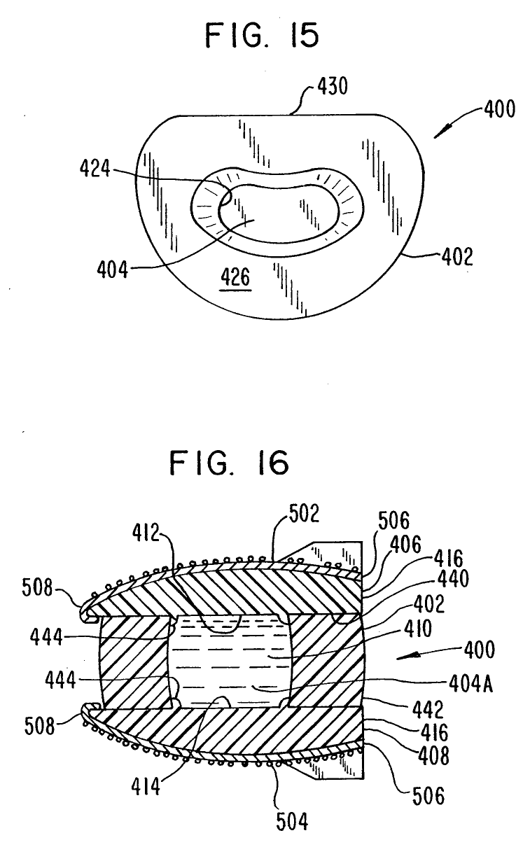

An embodiment of the total disk prosthesis is illustrated in FIGS. 6-16.

The illustrated embodiment of the total disk prosthesis comprises a disk core 400, upper and lower transition plates 406 and 408, and metal endplates 502 and 504. The disk core 400 comprises a polymer annulus 402 surrounding a nucleus cavity 404. The polymer annulus 402 has a cross-section generally resembling the cross-section of the intact natural annulus fibrosus. Its dimensions are designed to replace the natural annulus fibrosus in a particular patient. Accordingly, the polymer annulus 402 will have a transverse dimension ranging from about 2.5 cm to about 4.0 cm, and an antero-posterior dimension ranging from about 1.4 cm to about 3.0 cm. The thickness of the polymer annulus 402 is selected such that the overall thickness of the total disk prosthesis, when implanted, will provide substantially the same intervertebral spacing in the recipient as existed before the degeneration of the natural intervertebral disk, or at least such intervertebral spacing as will alleviate the symptoms produced by the degeneration of the natural intervertebral disk. Typically, the thickness, from upper surface to lower surface, of the polymer annulus 402 will range from about 0.4 cm to about 1.2 cm. The nucleus cavity 404 in the center of the polymer annulus 402 has a transverse cross-section generally conforming to the cross section of the intact natural nucleus pulposus. The nucleus cavity 404 is filled with a biocompatible incompressible material 410, which can be a fluid, such as a biocompatible oil, or a soft biocompatible polymer. The central cavity 404 occupies about 20% to about 80% of the volume of the polymer core 400 and the upper and lower contact areas 412, 414 with the transition plates 406 and 408 are flat and are centered midway between the anterior and posterior borders 416 and 418 of the transition plates 406 and 408, and midway between the lateral borders 420 and 422 of the transition plates 406 and 408. The upper and lower ends of the nucleus cavity 404 have a transverse cross section that is discoid in shape. The transverse cross section of the nucleus cavity 400 at the waist region 424 is about 30% to about 80% of the transverse cross sectional area of the upper and lower ends of the nucleus cavity 404. The nucleus cavity 404 is sealed by the transitional plates 406 and 408 which are sealed to the upper and lower surfaces 426, 428 of the polymer annulus 402 by molding thereto or by a suitable biocompatible adhesive.

In an alternate embodiment illustrated in FIG. 16, the nucleus cavity 404A may have generally vertical walls to form a generally cylindrical cavity with a discoid cross section, wherein the region generally midway between the upper and lower ends thereof does not have a pronounced waist shape.

The nucleus cavity 404 may be filled with a fluid, such as a biocompatible oil, or a soft or liquid polymer material. Such a polymer material may have the same general chemical composition as the polymer that forms the annulus 402 or it may be a chemically different material. For example, if the annulus is made from a polycarbonate polyurethane blend with a durometer of A70-A90, there is no soft grade of such a copolymer currently commercially available having a durometer less than A70, which might be used to fill the nucleus cavity 404. Therefore, for such an annulus 402, a chemically different kind of polymer, having a durometer less than A70, has to be used for filling the nucleus cavity 404, e.g., a silicone-based polymer.

The polymer annulus 402 is preferably made from a biocompatible polymer having a durometer in the range of about A70-A90. A preferred polymer for forming the polymer annulus 402 is a biocompatible polycarbonate-polyurethane blend. The outer perimeter of the polymer annulus 402 is discoid in shape, and the inner wall forms the nucleus cavity 404. Preferably, the nucleus cavity 404 has an hourglass or dumbbell shape. The volume of the polymer annulus 402 may vary in the range of about 20% to about 80% of the volume of the total polymer core, depending on the hardness of the polymer annulus and the hardness of the material filling the nucleus cavity 404. A polymer core 400 constructed with a nucleus cavity 404 having a volume of about 20-50% of the total volume of the polymer core 400 and filled with an incompressible fluid, and a polymer annulus 402 having a volume of about 50-80% of the total volume of the polymer core 400 and having an e-value of about 3-16 Mpa, provides biomechanical characteristics in compression, compression bending, and torsion generally equivalent to those of a natural intervertebral disk in the lumbo-sacral region of the spine. (A fluid material has no e-value.) A polymer core 400 with a nucleus cavity 404 having a volume of about 20-50% of the total volume of the polymer core 400 and filled with a soft polymer having an e-value of about 1-4 Mpa, and polymer annulus 402 having a volume of about 50-80% of the total volume of the polymer core 400 and having an e-value of about 4-16 MPa, provides biomechanical characteristics to those of the annulus with a fluid-filled core. Typically, a polymer core 400 having a central cavity 404 filled with an incompressible fluid provides better creep behavior than a polymer core having a central cavity filled with a polymer that is softer (lower e-value) than the polymer of the polymer annulus 402. Consequently such a polymer core 400 is a preferred embodiment.

The transitional endplates 406 and 408 are preferably made from a relatively very hard biocompatible polymer, such as a polycarbonate-polyurethane blend having a durometer hardness in a range of about A100-D70, and capable of being molded to the polymer annulus 402. The polymer endplates 406 and 408 have generally the same discoid transverse shape as the polymer annulus 402, but also incorporate a posterior tongue extension 432 and 434 beyond the posterior margin 430 of the annulus 402.

The outer surfaces 436 and 438 of the transitional plates, i.e., the surfaces facing the vertebral bone, are convex toward the endplates 502, 504. The inner surfaces 440, 442 of the transitional plates 406 and 408, facing the polymer annulus 402, are substantially flat to match the flat upper and lower surfaces of the polymer annulus 402, and are sealed to the surfaces of the polymer annulus 402 by conventional procedures such as molding or adhesive bonding. Preferably, the inner surfaces 440, 442 of the transitional plates 406, 408 are molded to the upper and lower surfaces 426, 428 of the polymer annulus 402.

One or both of transitional endplates 406, 408 may have an annular raised projection 444 (shown in cross-section in FIG. 16) on the surface facing the polymer annulus 402, that fits within the inner wall of the polymer annulus at the upper and/or lower surface thereof, to provide alignment between the polymer annulus 402 and the transitional plates 406, 408, and make a stronger and/or more secure seal. Such a projection will stabilize the interface between the annulus and the transition plate, especially in torsion and shear.

The posterior parts of the transitional plates are relatively thin, i.e, having a thickness in the range of about 1-3 mm, and the anterior parts of the transitional plates are somewhat thicker, i.e., in a range of about 4-7 mm. This difference in thickness at the anterior edges 416 and posterior edges 418 of the transitional plates 406, 408 will provide a lordotic angle 448 for the disk prosthesis (as can be seen, e.g., in FIG. 11) that can be tailored to an individual patient.

The endplates 502 and 504 of the disk prosthesis are made of any suitably strong and biocompatible material. Preferably the endplates 502 and 504 are made of a metal such as titanium, stainless steel, or Cr—Co alloy. The endplates typically have a uniform thickness. The upper and lower metal endplates 502, 504 of the disk prosthesis of the invention are convex toward the vertebral bone. The maximum depth of the convexity (vertex 516) is located on the midline between the lateral margins of the endplates in the coronal (right-left) plane, and is located about 60% posteriorly from the anterior margin of the plate in the sagittal (anterior-posterior) plane. The height of the convexity is typically about 1.5 mm-2.5 mm for the upper endplate 502 and about 0.6 mm-2.0 mm for the lower endplate 504.

The inner surface 514 of each endplate is preferably highly polished for smooth contact with the outer surface of the adjacent transitional endplate. The outer surface 512 of each endplate is preferably provided with a porous texture for bone ingrowth.

The posterior margin 508 of each endplate has an extension 522 curved toward or extending toward the transitional endplate to form a groove to receive the posterior margin 418 of the transitional plate, which extends beyond the posterior wall of the polymer annulus 402, in a “tongue and groove” engagement.

The anterior midline of one or both of the metal endplates 502, 504 has a fin 518 projecting toward the vertebral bone. This fin 518 is engaged in a cut or recess made in the vertebral bone at the anterior midline of the vertebral endplate. The fin 518 of each main metal endplate 502, 504, is double-walled, creating a slot 520 for receiving a mating fin 612 of the anterior extension plate 602, as discussed below.

Each anterior horizontal extension plate 602 is preferably made of the same material, e.g., metal, as the main metal endplate, and has generally the same thickness. Each horizontal extension plate has a posterior margin 606 that matches the horizontal curvature of the anterior margin 506 of the main metal plate. The anterior margin 604 of the extension plate is also curved to provide an antero-posterior depth at the midline of the prosthesis. Consequently, the horizontal extension plate 602 has its greatest antero-posterior dimension at the midline, and each side tapers from the anterior margin 604 toward the lateral-posterior margin 606. Each horizontal extension plate has a curved perpendicular plate 610 extending away from the adjacent vertebra along the curved posterior margin 606 of the extension plate 602. The curved perpendicular plate 610 matches the curvature and thickness of the anterior margin 416 of the transitional plates 406, 408. The perpendicular curved plate 610 may be provided with holes for screws that are driven into the anterior margin 416 of the transitional plate 406, 408 or introduced into threaded holes formed in the anterior margin 416 of the transitional plate. Typically two screw holes 620 are provided in each curved perpendicular curved plate 610, one on each side of the midline.

In the illustrated embodiment an endplate 502, its corresponding extension plate 602, and the adjacent transition plate 406, are provided with sleeves mounted on their lateral margins that are aligned, when the plates are assembled, to receive fastening screws 526.

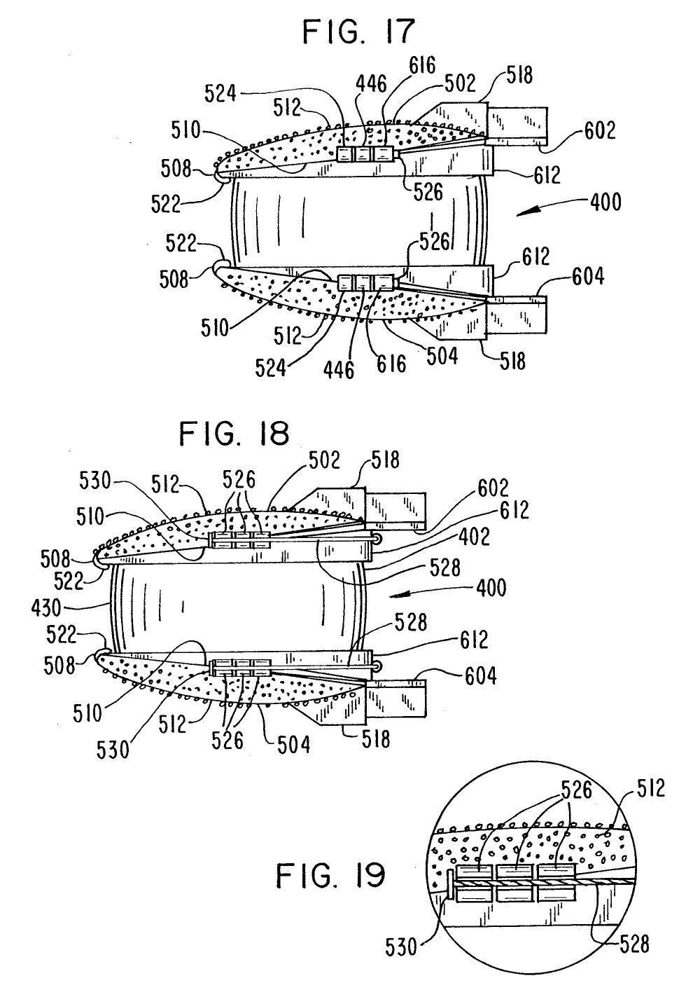

Alternatively, instead of using screw sleeves and screws, an endplate 502, transition plate 406, and extension plate 602 can be fastened together as shown in FIG. 18, and the detail FIG. 19, using a wire or cable 528, having a T-end 530 or equivalent end-stop structure, threaded through slotted sleeves 526, 448, and 622, and tightened by twisting or other conventional procedure such as the use of a conventional tightening device indicated schematically at 532.

A curved perpendicular plate 610 of a horizontal extension plate 602 may also have a resilient or spring appendage (not shown) for a snap-fit engagement with a recess formed in the anterior wall of the transition plate 406, 408.

In an alternative embodiment the main metal endplate may be made as a one-piece structure having a posterior extension to provide a grove for receiving the posterior margin of the transitional plate and resilient or elastic appendages at the anterior margin and at selected positions along the lateral margins to provide a snap-fit engagement with corresponding recess and/or groves in the anterior and/or lateral margins of the transitional plate.

In such an embodiment wherein the transitional plate is snap-fitted to the metal endplate, it may be further secured by providing slots in the snap-fit appendages to receive a tightening cable. Such a tightening cable has an end-stop structure extending transverse to the cable at the end thereof that will secure each end of the cable within a slot in a snap-fit appendage. The cable is then placed in the slots of the appendages and tightened by a forming a knot or by twisting, crimping, or by other conventional self-locking mechanism, typically located generally in the anterior portion of the total disk prosthesis.

Alternatively or additionally, a metal endplate and corresponding transitional plate and anterior extension plate may be fixed together with lateral appendages arranged for fastening with screws. In such an embodiment, appendages, e.g., sleeves, having through-holes for receiving assembly screws and threaded holes for accepting the threaded ends of the screws are arranged to be in line when the endplate, transition plate and extension plate are properly aligned, whereupon assembly screws are inserted and tightened to fix the plates firmly together. For, example, sleeves may be provided on the antero-lateral aspect of the metal endplate and the transitional plate and on the posterior-lateral corner of the extension plate, oriented and positioned so that the screw-holes will be aligned when the plates are properly assembled. Alternatively, such sleeves or similar appendages can be provided with slots to permit insertion of a wire or cable for fixation of the plates as indicated above.

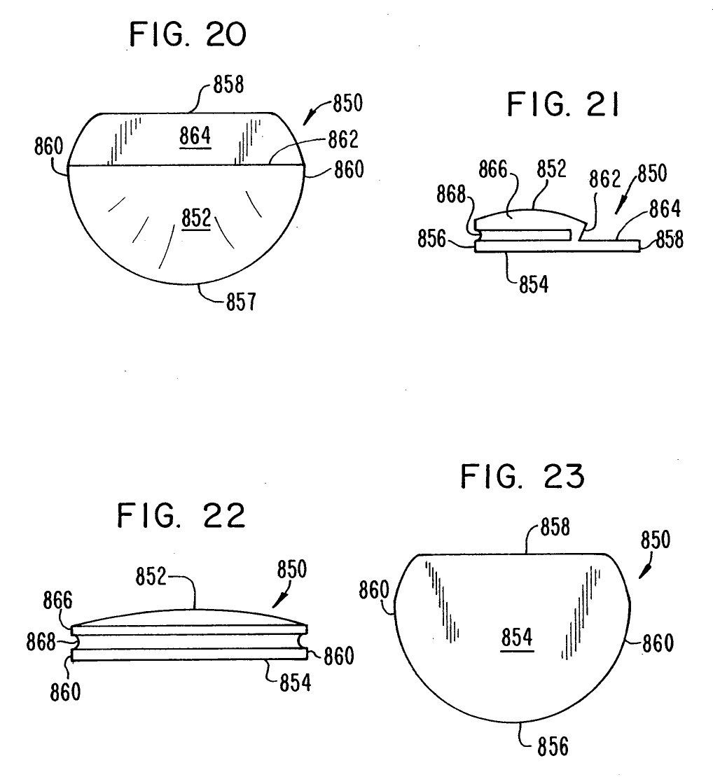

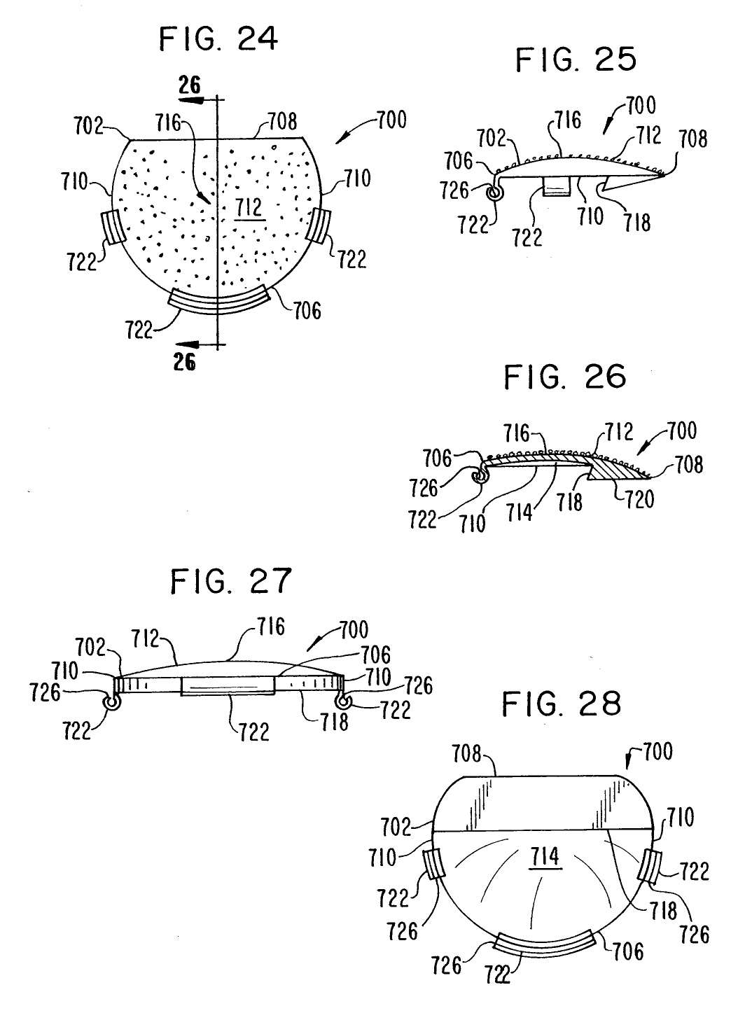

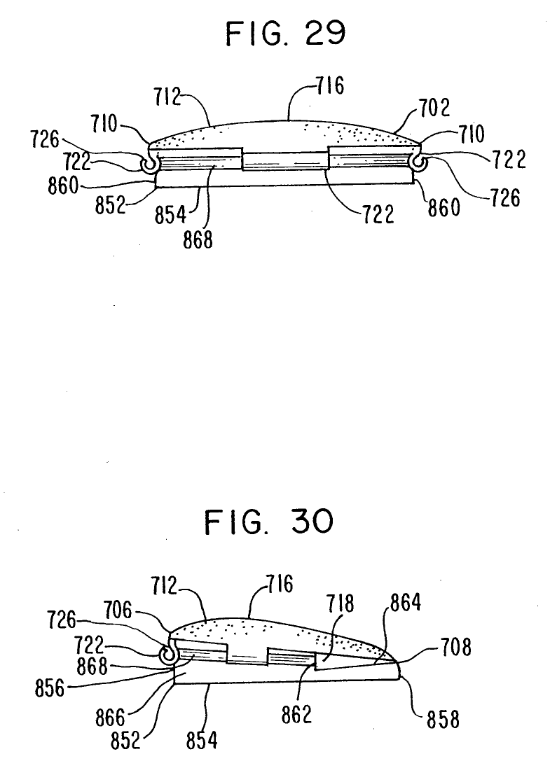

Another embodiment 700 of the transition plate-endplate structure utilizing a “step-cut” posterior portion is illustrated in FIGS. 20-30. In this embodiment the transition plate 850 (FIGS. 20-23) having an anterior margin 856, a posterior margin 858, and lateral margins 860 is provided with a step 862 extending between the lateral margins 860. The step 862 may be undercut as shown in FIG. 21. A recessed posterior portion 864 of the outer surface extends from the step 864 to the posterior margin 858 of the transition plate 850. The sidewall 866 has a peripheral groove 868 to receive the snap appendages 722 of the outer endplate 702, as discussed below.

The endplate 702 that is fastened to the transition plate 850 has an anterior margin 706, a posterior margin 708 and lateral margins 710. The outer surface 712 of the endplate 702 has a textured, e.g., porous, surface for bone ingrowth to assure good fixation to the vertebral endplate. The inner surface 714 is provided with a step 718 that engages a corresponding step 862 on the transition plate 850. A generally planar posterior surface 720 contacts the planar posterior surface 864 of the transition plate 850. The step 718 preferably has a reverse bevel as shown to engage the correspondingly reverse-beveled step 862 of the transition plate 850. Snap appendages 722 fit into peripheral groove 868 of transition plate 850 to fasten the endplate 702 to the transition plate 850. Slots 726 in appendages 722 are provided to receive a tightenable cable for additional security as illustrated in the embodiment shown in FIGS. 18 and 19. The assembly 700 comprising endplate 702 and transition plate 850 can be used in place of the similar assemblies shown, e.g., in FIGS. 17 and 18, to form the upper and lower portions of a total prosthesis such as illustrated therein.

The invention having been described above in terms of certain embodiments, it will be apparent to those skilled in that that many changes and alterations can be made without departing from the spirit or essential characteristics of the invention. All embodiments incorporating such changes or alterations are intended to be included within the invention. The present disclosure is therefore to be considered as illustrative and not restrictive, the scope if the invention being indicated by the appended claims, and all changes which come within the meaning and range of equivalency are intended to be included therein.

1. A prosthetic implant for replacing a nucleus pulposus of an intervertebral disk comprising:
an upper endwall and a lower endwall, each of said endwalls having a discoid cross-section and a periphery, and having an antero-posterior diameter and a transverse diameter, said antero-posterior diameter being greater than said transverse diameter; and
an hourglass-shaped sidewall connecting said peripheries of said upper endwall and said lower endwall; whereby an interior volume is enclosed between said upper endwall, said lower endwall and said sidewall;
said interior volume being filled with a substantially incompressible liquid or soft plastic material.

2. The prosthetic implant of claim 1 wherein said interior volume is filled with an aqueous normal saline solution.

3. The prosthetic implant of claim 1 wherein said interior volume is filled with a biocompatible oil.

4. The prosthetic implant of claim 1 wherein said interior volume is filled with a synthetic hyaluronic acid/proteoglycan composition.

5. (canceled)

6. The prosthetic implant of claim 1 wherein said interior volume is filled with a soft biocompatible synthetic polymeric material having a modulus in a range of 0 Mpa to about 1 Mpa.

7. The prosthetic implant of claim 1 wherein each of said upper and lower endwalls and said sidewall is made of biocompatible synthetic polymeric material.

8. The prosthetic implant of claim 7 wherein each of said upper and lower endwalls and said sidewall is made of biocompatible synthetic polymeric material having a durometer hardness in a range of A80 to D65.

9. The prosthetic implant of claim 7 wherein said biocompatible synthetic polymeric material is a polycarbonate-polyurethane blend.

10. The prosthetic implant of claim 9 wherein said polycarbonate-polyurethane blend has a durometer hardness in the range of A80 to D65.

11. The prosthetic implant of claim 1 wherein said endwalls have a thickness greater than a thickness of said sidewall.

12. The prosthetic implant of claim 1 wherein said biocompatible polymeric material of each endwall has a durometer hardness greater than a durometer hardness of said biocompatible polymer of said sidewall.

13-22. (canceled)

23. The prosthetic implant of claim 7 wherein said sidewall is made of a softer synthetic polymer than said endwalls.

24. The prosthetic implant of claim 7 wherein said sidewall is made of a thinner material than said endwalls.

25. The prosthetic implant of claim 1 wherein each of said endwalls has an area in a range of about 30% to about 60% of an area of a vertebral endplate which it is intended to contact.

26. The prosthetic implant of claim 1 wherein said internal volume has a narrowest transverse cross-sectional area in a range of about 20% to about 80% of a transverse cross-sectional area of said upper endwall.

27. The prosthetic implant of claim 1 wherein said internal volume has a narrowest transverse cross-sectional area in a range of about 20% to about 80% of a transverse cross-sectional area of said lower endwall.

28. The prosthetic implant of claim 1, additionally comprising at least one stabilizing cord attached to said implant.

29-32. (canceled)

33. A total prosthesis for replacing the entire human intervertebral disk, comprising:
a core comprising an annulus surrounding a central cavity, said annulus having upper and lower and side surfaces and made of a first biocompatible polymeric material and being shaped and sized to approximate the annulus fibrosus of a natural intervertebral disk, the first biocompatible polymeric material being an elastomer having a elastic modulus approximating that of the annulus fibrosus of the natural human intervertebral disk;
upper and lower transition plates affixed respectively to the upper and lower surfaces of the annulus the upper and lower transition plates being made of a second biocompatible polymeric material having a durometer hardness greater than that of the first biocompatible polymeric material; and
upper and lower endplates adapted to contact adjacent vertebrae and affixed respectively to the upper and lower transition plates;
wherein said central cavity has an hourglass shape.

34. The total prosthesis of claim 33, wherein said first biocompatible polymeric material is a first elastomeric synthetic polymeric material.

35. The total prosthesis of claim 34, wherein said first elastomeric synthetic polymeric material comprises a first polycarbonate-thermoplastic polyurethane blend.

36. The total prosthesis of claim 34, wherein said first elastomeric synthetic polymeric material has a durometer hardness in a range of about Shore A70 to about Shore A90.

37. (canceled)

38. The total prosthesis of claim 33, wherein said second biocompatible polymeric material comprises a second elastomeric synthetic polymeric material.

39. The total prosthesis of claim 38, wherein said second elastomeric synthetic polymeric material comprises a second polycarbonate-thermoplastic polyurethane blend.

40. The total prosthesis of claim 38, wherein said second elastomeric synthetic polymeric material has a durometer hardness in a range of about Shore A100 to about Shore D65.

41. (canceled)

42. The total prosthesis of claim 33, wherein said central cavity has a volume comprising about 20% to about 50% of the volume of said polymer core.

43. The total prosthesis of claim 33, wherein said annulus has a volume comprising about 50% to about 80% of said polymer core.

44. The total prosthesis of claim 33, wherein said cavity is filled with an incompressible liquid.

45. The total prosthesis of claim 33, wherein said cavity is filled with a biocompatible polymeric material having an e-value of about 1-4 megapascals.

46. The total prosthesis of claim 33, wherein each of said transition plates are molded to said upper and lower surfaces of the annulus.

47-49. (canceled)

50. The total prosthesis of claim 33, wherein each of said transition plates has a domed outer surface, and each of said endplates has an inner surface shaped to contact the domed outer surface of the corresponding transition plate.

51-58. (canceled)

59. The total prosthesis of claim 33, wherein at least one of said endplates comprises a main endplate and an anterior extension plate.

60. The total prosthesis of claim 59, wherein said anterior extension plate is provide with a fin upstanding from an outer surface thereof and adapted to interact with said fin on said main endplate.

61. The total prosthesis of claim 59, wherein said anterior extension plate is provided with a wall extending generally perpendicular to an inner surface of said anterior extension plate and adapted to contact an anterior edge of the corresponding transition plate.

62. The total prosthesis of claim 59, wherein said main endplate, the corresponding transition plate, and said anterior extension plate are each provided with sleeves at lateral edges thereof adapted to receive screws cooperating with said sleeves to fasten said main endplate, said corresponding transition plate and said anterior extension plate together.

63. The total prosthesis of claim 59, wherein said main endplate, the corresponding transition plate, and said anterior extension plate are each provided with appendages at lateral edges thereof adapted to receive a tightening cable to fasten said main endplate, said corresponding transition plate and said anterior extension plate together.

64. The total prosthesis of claim 33, wherein at least one said transition plate is provided with a recess having a forward wall located at a distance from a posterior edge of said transition plate and extending from said forward wall to said posterior edge.

65-69. (canceled)

70. The total prosthesis of claim 33, wherein at least one of said endplates is provided with at least one elastic appendage extending inwardly from a periphery of said endplate and adapted to fit into a corresponding recess in one of said transition plates to affix said at least one endplate to said one transition plate.

71. The total prosthesis of claim 70, wherein said at least one of said endplates is provided with a plurality of said elastic appendages.

72. The total prosthesis of claim 71, wherein said elastic appendages are provided with grooves for receiving a tightening cable.

73. The total prosthesis of claim 71, wherein at least one of said transition plates has an outer surface and an inner surface and a peripheral wall extending between said outer surface and said inner surface, and said peripheral wall is provided with at least one said recess for engaging said elastic appendage of said endplate.

74. The total prosthesis of claim 73, wherein said peripheral wall of said transition plate is provided with a peripheral groove for receiving said appendages.

75. The total prosthesis of claim 33 wherein each of said endplates has an area in a range of about 30% to about 100% of a vertebral endplate which it is adapted to contact.

76. The total prosthesis of claim 33 wherein each of said endplates has an area in a range of about 30% to about 80% of a vertebral endplate which it is adapted to contact.

77. The prosthetic implant of claim 7 wherein said biocompatible synthetic polymeric material comprises polycarbonate and polyurethane.

78. The prosthetic implant of claim 77 wherein said biocompatible synthetic polymeric material has a durometer hardness in the range of A80 to D65.

79. The total prosthesis of claim 34, wherein said first elastomeric synthetic polymeric material comprises polycarbonate and polyurethane.

80. The total prosthesis of claim 38, wherein said second elastomeric synthetic polymeric material comprises polycarbonate and polyurethane.
